(19) United States
(12) Patent Application Publication
(10) Pub. No.: US 2014/0100490 A1
(43) Pub. Date: Apr. 10, 2014

(54) INHALANT DELIVERY SYSTEM FOR MASSAGE APPARATUS

(71) Applicant: Eric Mark CHELGREN, Ottumwa, IA (US)

(72) Inventor: Eric Mark CHELGREN, Ottumwa, IA (US)

(21) Appl. No.: 13/826,390

(22) Filed: Mar. 14, 2013

Related U.S. Application Data
(60) Provisional application No. 61/712,080, filed on Oct. 10, 2012.

(51) Int. Cl. A61H 37/00; A61M 11/00; A61M 16/20; A61M 16/00; A61M 16/08

(52) U.S. Cl. CPC A61H 37/00; A61M 16/0057; A61M 16/0875; A61M 16/20; A61M 11/00
USPC 601/24; 128/202.18

(57) ABSTRACT

A inhalant delivery system for supplying oxygen or another harmless gas to a user positioned on a massage apparatus such as a massage table or massage chair. A plenum having exit openings conforms generally to the inner periphery of a headrest of the massage apparatus such that the exit openings direct the oxygen or other harmless gas toward the nostrils of the user. The plenum is coupled to a supply of the gas. An optional infusion port may be included in the plenum to allow a fragrance or other additive to be infused in the stream of gas passing through the plenum.

### CROSS REFERENCE TO RELATED APPLICATIONS

This application claims priority under 35 USC §119 from copending provisional patent application entitled INHALANT DELIVERY SYSTEM FOR MASSAGE APPARATUS, Ser. No. 61/712,080 filed Oct. 10, 2012. The disclosure of provisional patent application Ser. No. 61/712,080 is hereby incorporated in its entirety.

### BACKGROUND TO THE INVENTION

Massage therapy is accepted for promoting wellbeing and relaxation through reduction of stress, pain and muscle tension. Massage apparatus has become more widely used because of the increasing awareness of the public of the salutary effects of body massage. Such massage apparatus includes massage tables and massage chairs, each of which includes a headrest to support the head of the person receiving a massage so that when lying prostrate on a massage table, or leaning forward in a massage chair, the person receiving the massage may rest his or her face on the headrest with the nostrils, eyes and mouth unobstructed. Such a headrest typically includes a horseshoe shaped cushion which will support the periphery of the face while allowing the eyes, nostrils, mouth and chin of the user to protrude into the void in the cushion.

It is known that use of augmented oxygen during intense exercise is useful to reduce lactic acid in muscles to prevent muscles from becoming fatigued. It is also widely accepted that administration of oxygen after exercise will improve the recovery time needed following intense exercise or work.

Because treatment through massage therapy is intended to promote relaxation and health, and because augmented administration of oxygen to a person promotes muscle recovery after exercise, it would be useful to have apparatus to supply additional oxygen to a person who is receiving a massage following a period of exercise. Administration of oxygen during a massage would increase the benefits of the massage therapy if performed in a convenient and passive manner. Apparatus to conveniently provide oxygen to a person receiving a massage would be desirable.

### SUMMARY OF THE INVENTION

The present invention provides an improvement to massage apparatus in order to enhance the experience of the person resting on the massage table or sitting in the massage chair. Specifically, the invention provides apparatus to provide a flow of O₂ or other desirable gas toward the face and nostrils of the user resting upon the massage apparatus. Optionally, a scent or aroma component may be infused into the stream of O₂ before the stream exits openings alongside the face of the user.

The invention comprises a horseshoe shaped plenum supported by a planar base shaped and sized to fit between the face pillow cushion and the pillow support base on which the cushion is detachably supported. The base is formed in the shape of an inverted U or horseshoe and may conform generally to the shape of the pillow support base. The base includes a plenum following the inner periphery of the base, the plenum having openings on the opposing legs of the plenum which are located along the inner periphery of the base. The openings direct flow of gas slightly upward and toward the face of a user.

The first object of the invention is to provide apparatus to selectively supply an invigorating breeze toward the face and nostrils of a user of a massage table who has rested his or her head upon the horseshoe shaped pillow of the massage apparatus.

### BRIEF DESCRIPTION OF THE DRAWINGS

FIG. 1 is a perspective of a massage apparatus modified according to the present invention.

FIG. 2 is an exploded view of the face pillow assembly of a massage apparatus which has been modified according to the present invention.

FIG. 3 is a top left perspective view of the base plate of the invention.

FIG. 4 is a bottom left perspective view of the base plate of the invention.

FIG. 5 is a top view of the base plate of the invention.

FIG. 6 is a cross section taken along line 6-6 of FIG. 5.

FIG. 7 is an enlargement of the segment of the plenum of the base shown in circle 7 of FIG. 6.

### DETAILED DESCRIPTION

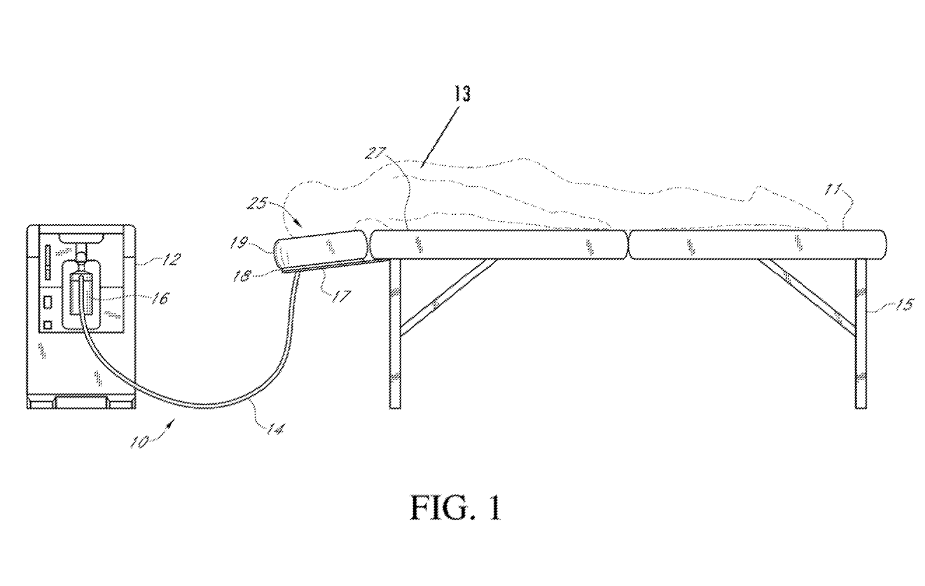

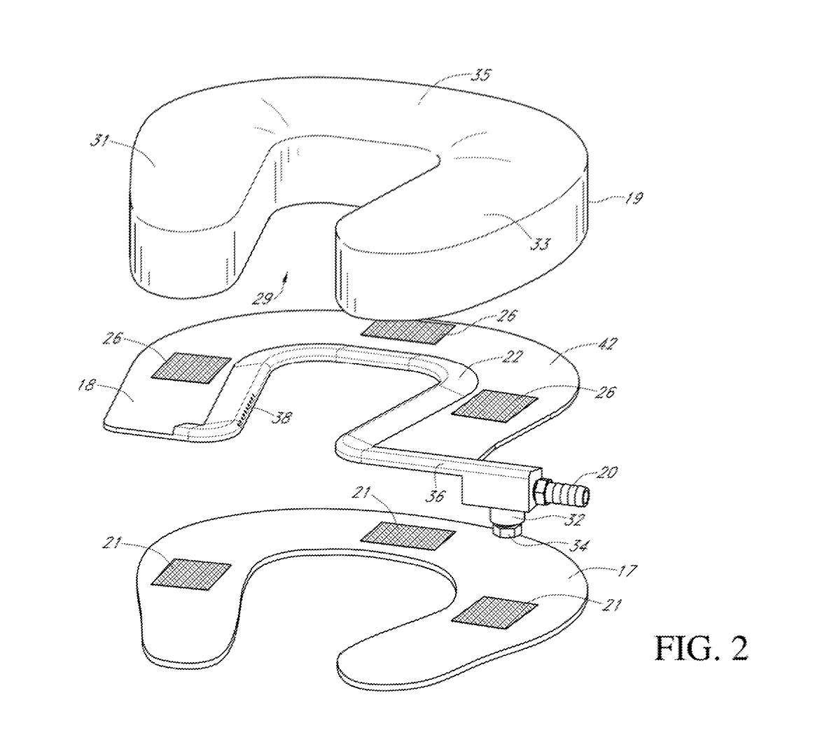

FIG. 1 shows a typical massage table 11 which has been equipped with the system invention 10. An inhalant source such as an oxygen concentrator 12 is coupled by tubing 14 to the base plate 18 of the invention 10. A headrest assembly 25 is provided at a head end 27 of the table 11 so that a user 13 upon whom a massage is to be given may rest her head with her face protruding into the void 29 (see FIG. 2) of the pillow cushion 19 of the headrest assembly 25. Massage table 11 includes pillow support base 17 and pillow cushion 19 which are part of the standard headrest assembly 25 of a massage table 11 or of a massage chair (not illustrated). In the case of a massage chair, the user sits leaning forward with knees resting on a cushion and with the user's chest resting on a chest cushion and with her head resting on a headrest like headrest assembly 25.

Pillow cushion 19 is generally horseshoe shaped with opposing sides 31, 33 joined by curved interconnecting segment 35. A void 29 is defined by opposing sides 31, 33 and curved interconnecting segment 35. When used by a user, the user's face is received in the void 29 while the peripheral parts of the face are supported by the pillow cushion 19.

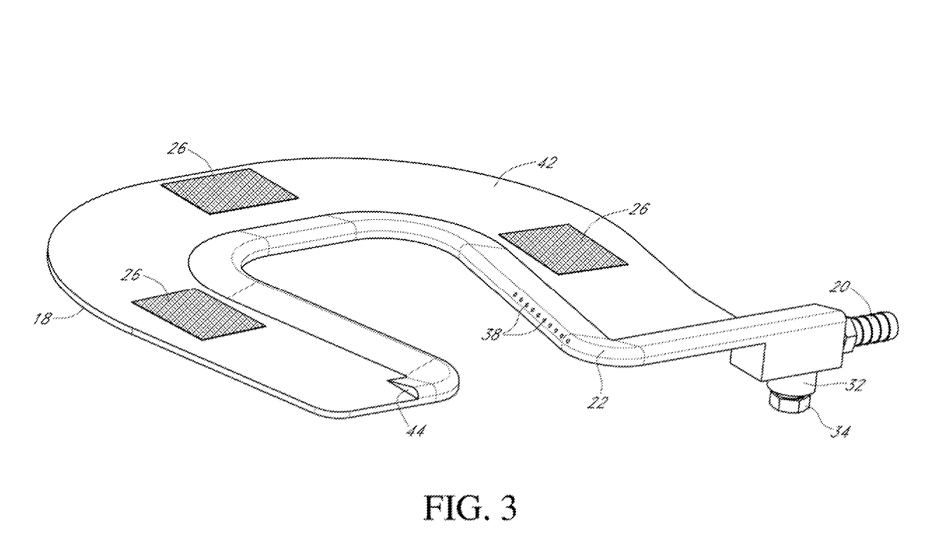

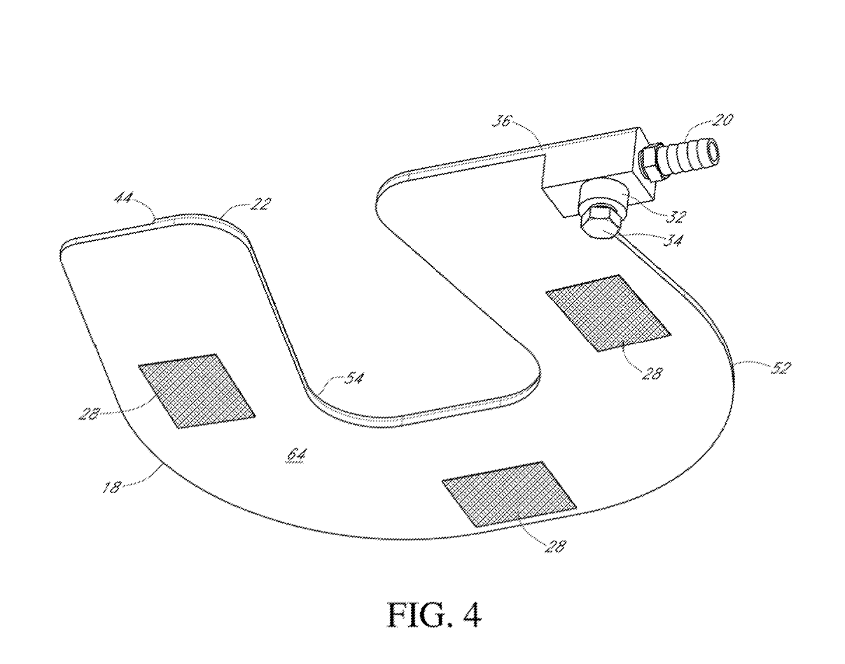

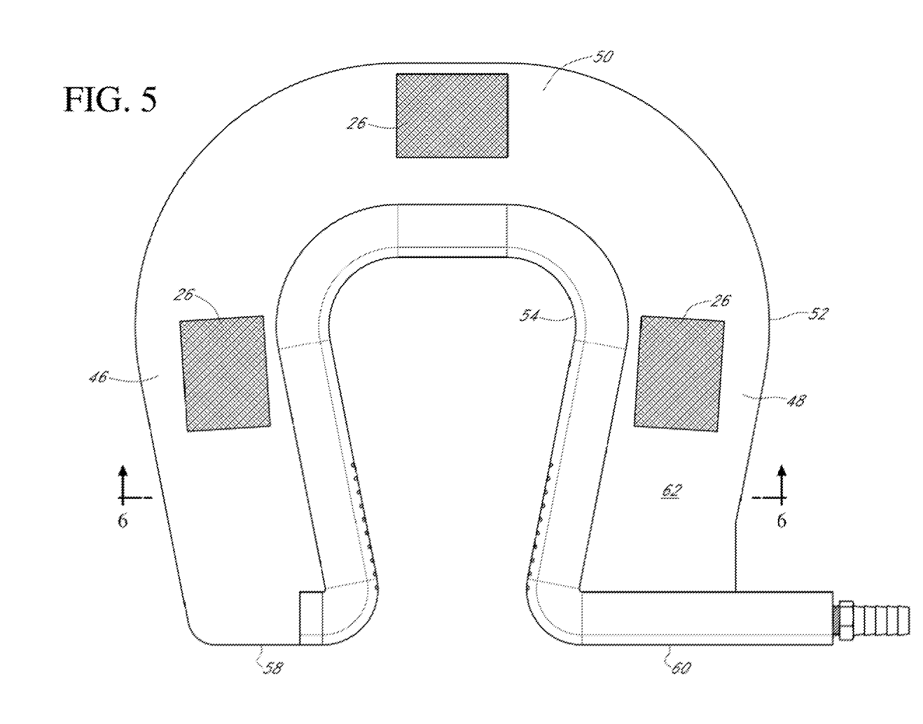

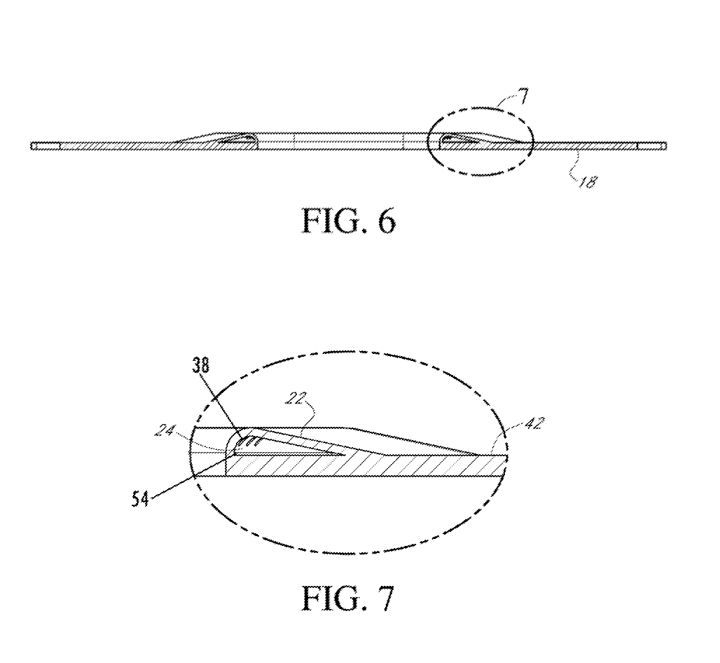

As seen in more detail in FIGS. 2-7, the invention 10 comprises a horseshoe shaped base plate 18 which is substantially planar so that it can be installed between the pillow support base 17 and the pillow cushion 19 which are part of the horseshoe-shaped headrest assembly 25 of the massage table 11 or of a massage chair (not illustrated). Base plate 18 is constructed of a rigid material such as metal or polymer composite.

The base plate 18 includes opposing legs 46, 48 which are joined by a curved element 50 and base plate 18 generally conforms to the shape of the pillow cushion 19 and the pillow support base 17 and may be received between them. The base plate 18 includes an outer periphery 52 and an inner periphery 54 along which a plenum 22 within the base plate 18 is provided. Plenum 22 may be non-circular in cross section and in the illustrated embodiment is tear drop shaped in cross section (see particularly FIG. 7) with the enlarged portion 24 of the plenum 22 disposed along the inner periphery 54 of the base plate 18. The plenum extends substantially the full length of the inner periphery 54 and is coupled at its intake end 36 to a coupling 20 with which plenum 22 is communicative and which may be joined to tubing 14 which is also coupled to an inhalant source, such as the oxygen concentrator 12 seen in FIG. 1. In the general case, the inhalant will be O₂, though other harmless gases may be employed at appropriate concentrations and durations.

Plenum 22 is closed at its second end 44. A shut off valve (not shown) may be provided between the coupling 20 and the plenum 22 if desired. In addition, an infusion port 32 may be provided near intake end 36 of plenum 22 which communicates with interior of the plenum 22 so that a scent or aroma or other desirable vapor may be infused into the stream of O₂ or other inhalant gas passing into plenum 22, as desired. It is to be understood that infusion port 32 would be used only when a scent or other vapor is to be introduced into the stream of inhalant gas. Infusion port 32 may comprise a scent receptacle 34 which may be provided with a wick (not shown) or other similar introduction element which extends into the stream of O₂ or other inhalant gas flowing past the infusion port 32. Other means of injecting scent or aromas may be used, such as an atomizer attached to infusion port 32 or tubing from an aroma source or scent source may be coupled to infusion port 32.

Plenum 22 is provided with plural exit ports 38 positioned along the inner periphery 54 along the opposing legs of 46, 48 of base plate 18, preferably near the free ends 58, 60 of legs 46, 48 respectively. Exit ports 38 communicate with the interior of plenum 22 and may be cut or drilled or otherwise formed so as to direct O₂ other inhalant gas exiting from exit ports 38 toward the center of void 29 so that a gentle inhalant breeze of harmless gas (e.g. O₂ or other desirable gas or vapor) is directed toward the face and particularly toward the nostrils of the user 13 of the massage table 11.

Typically, pillow cushion 19 fastens to pillow support base 17 by detachable fasteners such as hook and loop fasteners. Complementary hook and loop fasteners 26, 28 may be attached to top face 64 and bottom thee 66 of base plate 18 respectively so that base plate 18 may be detachably fastened between pillow cushion 19 and pillow support base 17.

The system invention 10 may be easily adapted for use with a massage chair having a headrest assembly constructed generally similar to headrest assembly 25. In the usual massage chair application, the headrest assembly allows a user seated in the massage chair to lean forward and to rest his or her head on a horseshoe pillow cushion with the user's face protruding into the void within the pillow cushion. The system invention 10 may be installed below the pillow cushion and upon a pillow support base generally similar to pillow support base 17 described above as an attachment to massage table 11. An inhalant gas such as O₂ may be delivered via exit ports 38 of plenum 22 to the face of the user resting his or her head on the pillow cushion. The inhalant gas may be scented or infused with aroma incorporated into the inhalant gas stream as it passes the infusion port 32. Therefore, the user of the massage chair may enjoy the gentle breeze of the inhalant gas as it is directed toward the face of the user.

What is claimed is:

1. Apparatus to supply a harmless inhalant gas to a user of a massage apparatus comprising
a base comprising a hollow plenum,
the base combinable to a headrest assembly of the massage apparatus adjacent a horseshoe shaped cushion thereof,
the plenum including at least one exit port disposed adjacent an inner periphery of the cushion,
the at least one exit port communicative with an interior of the plenum,
the plenum communicative with a source of the harmless inhalant gas,
wherein the harmless inhalant gas may pass along the plenum and may exit from the at least one exit port of the plenum.

2. The apparatus of claim 1 wherein
the plenum comprises multiple exit ports,
each of the exit ports positioned generally along the inner periphery of the cushion.

3. The apparatus of claim 1 wherein
the plenum further comprises an infusion port in communication the interior of the plenum,
wherein a second inhalant may be infused into a stream of the inhalant gas passing along the plenum.

4. The apparatus of claim 2 wherein
the base is generally horseshoe shaped,
the plenum disposed along an inner periphery of the base,
the plenum further comprising opposing legs and a curved segment interconnecting first ends of the legs,
the exit ports disposed along the opposing legs.

5. The apparatus of claim 4 wherein
the plenum is coupled to the source of harmless inhalant gas by a coupling,
a duct communicative between the source of harmless inhalant gas and the coupling.

6. The apparatus of claim 5 further comprising
a shut off valve along the duct.

7. The apparatus of claim 6 wherein
the harmless inhalant as is oxygen,
the plenum further comprises an infusion port in communication the interior of the plenum,
wherein a second inhalant may be infused into the oxygen passing along the plenum.

8. Apparatus to supply a harmless inhalant gas to a face of a user of a massage apparatus comprising
a plenum comprising an intake end coupled to a source of the inhalant gas,
the plenum disposed adjacent a headrest of the massage apparatus,
the headrest having a face receiving void for receiving the face of the user of the massage apparatus,
the plenum including exit ports disposed adjacent the face receiving void of the headrest,
wherein the harmless inhalant gas may be supplied to the plenum and may exit from the exit ports of the plenum toward the face of the user of the massage table.

9. The apparatus of claim 8 wherein
the plenum is generally horseshoe shaped,
the plenum comprising opposing legs interconnected at first ends thereof by a curved section,
the exit ports disposed along at least one of the opposing legs.

10. The apparatus of claim 9 wherein
the exit ports are disposed along an inner periphery of each of the opposing legs.

11. The apparatus of claim 8 wherein
the plenum includes an infusion port in communication therewith,
wherein a second inhalant may be infused into the inhalant gas in the plenum.

12. The apparatus of claim 8 wherein
the plenum is generally horseshoe shaped,
the plenum comprising opposing legs interconnected at first ends thereof by a curved section,
the exit ports disposed along an inner periphery of each of the opposing legs,
the plenum includes an infusion port in communication therewith,
wherein a second inhalant may be infused into the inhalant gas in the plenum.

13. The apparatus of claim 12 wherein
the plenum is coupled at an intake coupling to a duct coupled to a source of the inhalant gas,
the infusion port communicative with the plenum adjacent the intake coupling.

14. The apparatus of claim 13 wherein
the harmless inhalant gas is oxygen,
the source of the inhalant gas is an oxygen source,
a shut off valve is disposed along the duct.

15. Massage apparatus comprising
a platform adapted for a person to rest prostrate thereon,
the massage apparatus including a headrest assembly extending from the platform thereof,
the headrest assembly including a horseshoe shaped cushion,
the cushion adapted to receive and to support the head of the person,
the cushion including a void between opposing legs thereof,
the void adapted to receive the face of the person,
a plenum disposed beneath the cushion and having an inner periphery conforming with an inner periphery of the cushion,
the plenum including a hollow therein,
the plenum further comprising exit openings,
the exit openings communicative with the hollow of the plenum,
the hollow of the plenum communicative with a supply of an inhalant gas.

16. The massage apparatus of claim 15 wherein
the exit openings are disposed along an inner periphery of the plenum.

17. The massage apparatus of claim 15 wherein
the plenum including opposing legs,
the exit openings disposed along an inner periphery of at least one of the opposing legs of the plenum,
a base plate conforms to the periphery of the cushion,
the base plate including the plenum,
the headrest assembly including a support plate,
the base plate disposed between the cushion and the support plate.

18. The massage apparatus of claim 15 wherein
the plenum further comprises an intake port,
the intake port coupled to a duct communicative with the supply of inhalant gas,
the inhalant gas is oxygen,
the intake port communicative with the hollow of the plenum.

19. The massage apparatus of claim 18 wherein
the intake port comprises a wick extending into a stream of inhalant,
the wick communicative with a second inhalant contained in a receptacle.

20. The massage apparatus of claim 15 wherein
an atomizer is attached to an infusion port,
the infusion port communicative with the hollow of the plenum.
